(19) United States
(12) Patent Application Publication
(10) Pub. No.: US 2011/0054168 A1
(43) Pub. Date: Mar. 3, 2011

(54) METHOD FOR PREPARING ADENINE COMPOUND

(76) Inventors: Ayumu Kurimoto, Osaka-fu (JP); Wataru Katoda, Osaka-fu (JP); Kazuki Hashimoto, Osaka-fu (JP); Kazuhiko Takashi, Osaka-fu (JP)

(21) Appl. No.: 12/863,297

(22) PCT Filed: Jan. 16, 2009
(86) PCT No.: PCT/JP2009/050549

§ 371 Date: Jul. 16, 2010

(30) Foreign Application Priority Data
Jan. 17, 2008 (JP) 2008-007976

(51) Int. Cl. C07D 473/18

(52) U.S. Cl. 544/118; 544/276

(57) ABSTRACT

A method for preparing compound (4):

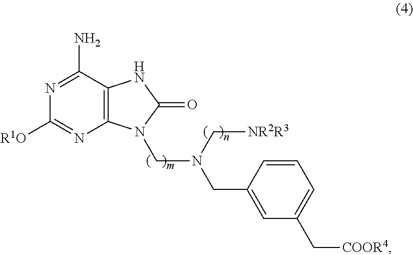

wherein m and n are independently an integer of 2 to 5, R¹ is C₁₋₆ alkyl group, R² and R³ are combined with an adjacent nitrogen atom to form pyrrolidine, morpholine, thiomorpholine, etc., and R⁴ is C₁₋₃ alkyl group, or a pharmaceutically acceptable salt, which is useful as a medicament, which comprises step (a) for preparing compound (2):

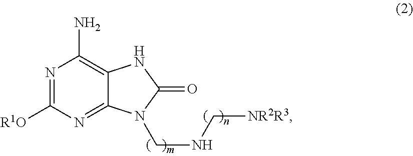

wherein m, n, R¹, R² and R³ are the same as defined above,
or its salt which comprises subjecting compound (1):

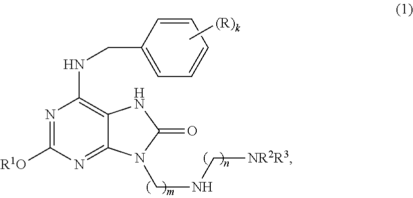

wherein k is an integer of 1 or 2, R is hydrogen atom, halogen atom, etc., or a salt thereof to debenzylation reaction,
and then
step (b) for preparing compound (4) or a pharmaceutically acceptable salt which comprises reacting compound (2) or its salt prepared in the above step (a) or salt thereof and compound (3):

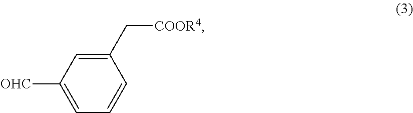

wherein R⁴ is C₁₋₃ alkyl group,
in the presence of a boron-containing reducing agent.

### TECHNICAL FIELD

The present invention relates to a method for preparing an adenine compound useful as a medicament, and to an intermediate for preparing it.

### BACKGROUND OF ART

An adenine compound represented by the following formula (4):

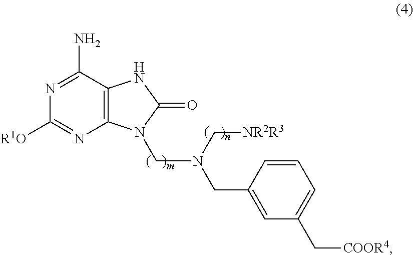

wherein R⁴ is C₁₋₃ alkyl group, and m, n, R¹, R² and R³ are the same as defined in compound (1) below,
is known to be useful as a medicament (See patent documents 1 and 2.).

It is described in patent document 2 that methyl (3-{[[3-(6-amino-2-butoxy-8-oxo-7,8-dihydro-9H-purine-9-yl)propyl](3-dimethyaminopropyl)amino]methyl}phenyl)acetate (included in compound (4)) is prepared by alkylating 6-amino-2-butoxy-9-{3-[(3-hydroxypropyl)amino]propyl}-7,9-dihydro-8H-purine-8-one which is prepared via 6-amino-9-(3-bromopropyl)-2-butoxy-7,9-dihydro-8H-purine-8-one, with methyl 3-bromomethylphenylacetate, and then reacting it with mesyl chloride and dimethylamine, successively, and so on.

Furthermore, it is described in patent document 1 that methyl (3-{[[4-(6-amino-2-butoxy-8-oxo-7,8-dihydro-9H-purine-9-yl) butyl] (3-morpholin-4-ylpropyl)amino]methyl}phenyl)acetate is prepared by reacting 9-(4-bromobutyl)-2-butoxy-8-methoxy-9H-purine-6-amine with 3-morpholinopropylamine to prepare 2-butoxy-8-methoxy-9-{4-[(3-morpholin-4-ylpropyl)amino]butyl)-9H-purine-6-amine, and then treating it with an acid, followed by alkylation with methyl 3-bromomethylphenylacetate.

However, as intermediates for preparation of the object compound (4), namely such compounds having an amino group at position 6, and that an oxo group at position 8 as 6-amino-9-(3-bromopropyl)-2-butoxy-7,9-dihydro-8H-purine-8-one and the like have the same pharmacological activities as ones of the compound (4), special attention must be paid to handling such compounds in the process for preparation of them.

As such it has been desired to develop for an effective method for preparing compound (4) without via such active intermediates.

In patent document 3 below, adenine compounds having benzylamino group on position 6 of purine nucleus are described, but there is no description herein on the method for preparing adenine compounds wherein benzyl group is utilized as a protective group for amino group on position 6 of purine nucleus.

Patent document 1: WO 2005/092893 gazette
Patent document 2: WO 2007/031726 gazette
Patent document 3: WO 00/043394 gazette

### DISCLOSURE OF THE INVENTION

### Problem to be Solved by the Invention

Problem to be solved by the invention is to provide a novel method for preparing an adenine compound represented by the formula (4) and a pharmaceutically acceptable salt thereof which is useful for a medicament.

### Means for Solving the Problem

The present inventors have been extensively studied for establishment for a novel method for preparing an adenine compound represented by the formula (4) or a pharmaceutically acceptable salt thereof and as a result the present invention has been completed.

Namely the present invention relates to methods for preparing compound (4) set forth in [1] to [10] below.

[1] A method for preparing a compound represented by the following formula (4):

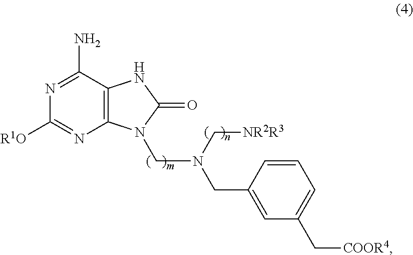

wherein m and n are independently an integer of 2 to 5, R¹ is C₁₋₆ alkyl group, R² and R³ are the same or different, and hydrogen atom, or C₁₋₆ alkyl group, or R² and R³ are combined with an adjacent nitrogen atom to form pyrrolidine, morpholine, thiomorpholine, piperidine, homopiperidine, piperazine or homopiperazine, and nitrogen atom of position 4 of said piperazine or homopiperazine may be substituted by C₁₋₄ alkyl group, and R⁴ is C₁₋₃ alkyl group,
or a pharmaceutically acceptable salt,
which comprises step (a) for preparing a compound represented by the following compound (2):

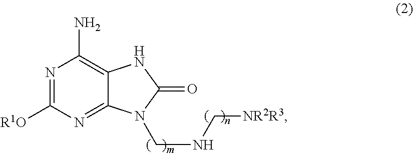

wherein m, n, R¹, R² and R³ are the same as defined above,
or its salt which comprises subjecting a compound represented by the following formula (1):

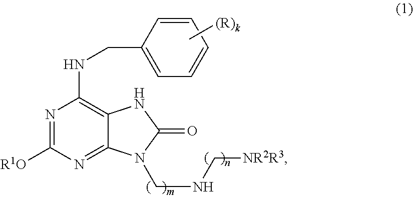

wherein k is an integer of 1 or 2, R is hydrogen atom, halogen atom, C₁₋₆ alkyl group, alkyloxy group or nitro group, and when k is 2, R is the same or different, and R¹, R², R³, m and n are the same as defined above, or a salt thereof to debenzylation reaction,
and then
step (b) for preparing compound (4) or its a pharmaceutically acceptable salt which comprises reacting compound (2) or its salt prepared in the above step (a) or a salt thereof and a compound represented by the formula (3):

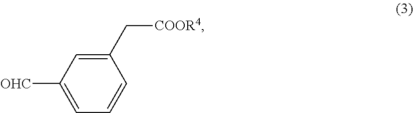

wherein R⁴ is C₁₋₃ alkyl group,
in the presence of a boron-containing reducing agent.
[2] The method according to above [1] wherein the debenzylation reaction is carried out by (a1) hydrogenation using hydrogen gas, formic acid or ammonium formate in the presence of a palladium-carbon catalyst or hydroxypalladium-carbon catalyst, or by (a2) debenzylation using alkyl chloroformate or substituted alkyl chloroformate.
[3] The method according to above [1] or [2] wherein the boron-containing reducing agent in step (b) is sodium triacetoxyborohydride.
[4] The method according to any one of above [1] to [3] wherein the method contains further process for preparing compound (1) or its salt, which comprises steps (c) for preparing a compound represented by the following formula (7):

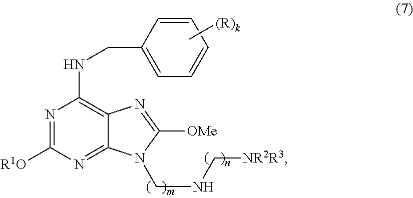

wherein k, m, n, R, R¹, R² and R³ are the same as defined above,
or a salt thereof,
which comprises reacting a compound represented by the formula (5):

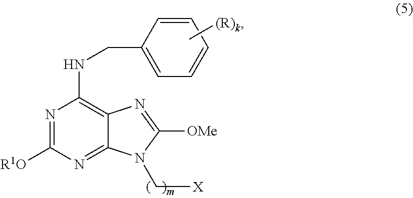

wherein X is chlorine atom, bromine atom or methanesulfonyloxy group,
R and R¹ are the same as defined above,
and a compound represented by the formula (6):

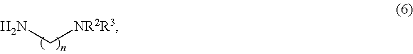

wherein n, R² and R³ are the same as defined above,
or a salt thereof,
and then
step (d) for preparing compound (1) which comprises treating compound (7) or its salt with an acid.
[5] The method according to any one of [1] to [3] wherein the method contains further process for preparing compound (1) or its salt, which comprises step (e) for preparing a compound represented by the following formula:

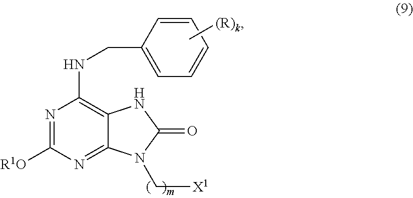

wherein k, m, R, R¹ and X¹ are the same as defined above,
which comprises treating a compound represented by the following formula (8):

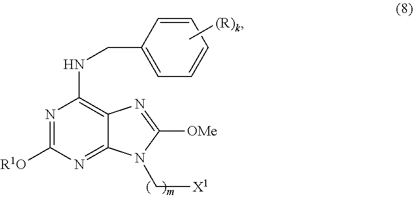

wherein X¹ is chlorine atom or bromine atom and k, m, R and R¹ are the same as defined above,
with an acid, and then
step (f) for preparing compound (1) or its salt which comprises reacting compound (9) obtained above with a compound represented by the formula (6):

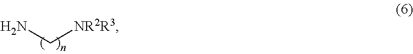

wherein n, R² and R³ are the same as define above,
or its salt.
[6] The method according to above [4] or [5] wherein the acid used in step (d) or (e) is an acid or acids selected from hydrochloric acid, hydrobromic acid, sulfuric acid, methanesulfonic acid and p-toluenesulfonic acid.
[7] The method according to any one of [4] to [6] wherein the method contains further process for preparing compound (8) or its salt, which comprises step (g) for preparing compound (8) which comprises reacting a compound represented by the formula (10):

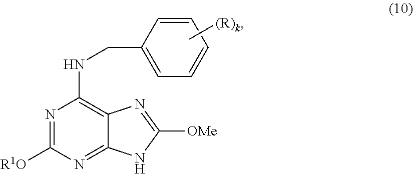

wherein k, R and R¹ are the same as defined above, or its salt with a compound represented by the formula (11):

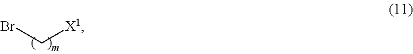

wherein m and X¹ are the same as defined above, in the presence of a base.
[8] The method according to any one of [4] or [6] wherein the method contains further process for preparing a compound represented by the following formula (15):

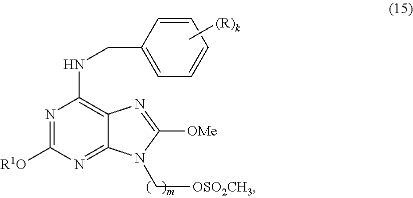

wherein k, m, R and R¹ are the same as defined above, or its salt, which comprises step (h) for preparing a compound represented by the following formula (13):

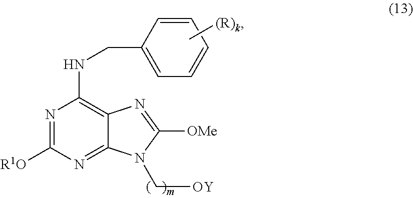

wherein Y is hydrogen atom or a protective group of hydroxy group, and k, m, R and R¹ are the same as defined above,
which comprises reacting a compound represented by the following formula (10):

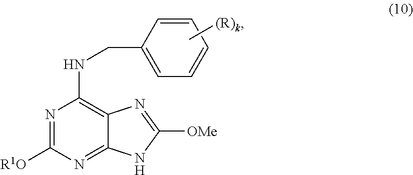

wherein k, R and R¹ are the same as defined above,
or its salt with a compound represented by the following formula (12):

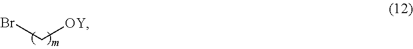

wherein Y and m are the same as defined above,
in the presence of a base,
and then step (i) for preparing a compound represented by the following formula (14):

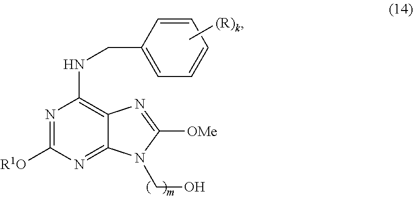

wherein k, m, R and R¹ are the same as defined above,
which comprises subjecting compound (13) wherein Y is a protecting group of hydroxy group to deprotection reaction,
and then step (j) for preparing compound (15) which comprises reacting compound (14) prepared by step (h) or (i) with methanesulfonyl chloride in the presence of a base.
[9] The method according to [7] or [8] wherein the method contains further process for preparing compound (10) or its salt, which comprises step (k) for preparing a compound represented by the following formula (18):

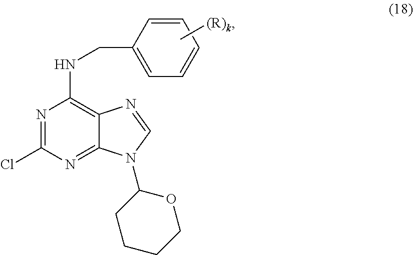

wherein k and R are the same as defined above,
which comprise reacting a compound represented by the following formula (16):

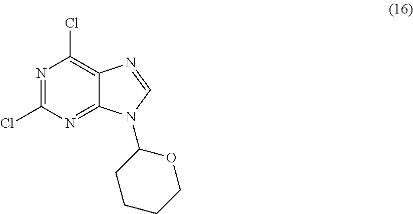

with a compound represented by the formula (17):

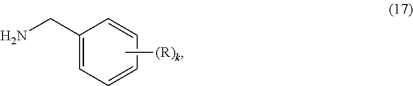

wherein k and R are the same as defined above,
or its salt, and then
step (1) for preparing a compound represented by the following formula (20):

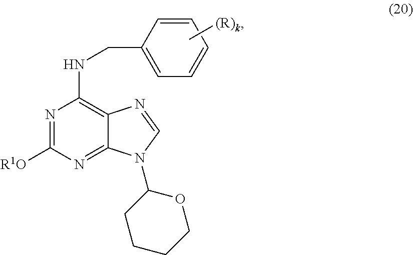

wherein k, R and R¹ are the same as define above,
which comprises reacting compound (18) prepared in step (k) with a compound represented by the following formula (19):

R¹—OH (19)

wherein R¹ is the same as defined above,
in the presence of a base,
and then step (m) for preparing a compound represented by the formula (21):

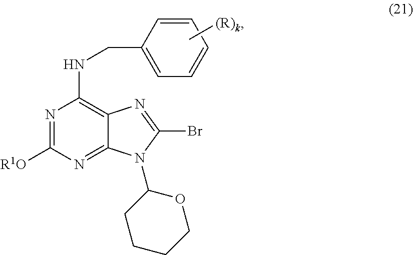

wherein k, R and R¹ are the same as defined above,
which comprises reacting compound (20) prepared in step (1) with bromine in the presence of sodium acetate or sodium phsphate, or in the absence of said salt and then step (n) for preparing a compound represented by the following formula (22):

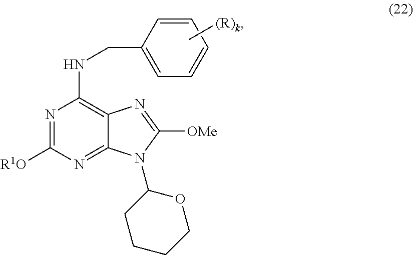

wherein k, R and R¹ are the same as defined above,
which comprises reacting compound (21) prepared in step (m) with methanol in the presence of a base, and then
step (o) for preparing compound (10) which comprises treating compound (22) prepared in step (n) with an acid.
[10] The method according to [9] wherein the acid used in step (o) is trifluoroacetic acid.

The present invention relates to new compounds set forth in the following [11] and [12], which are useful as an intermediate for preparing compound (4).

[11] A compound selected from compounds represented by following formulas (1), (5), (7), (9), (10) and (13):

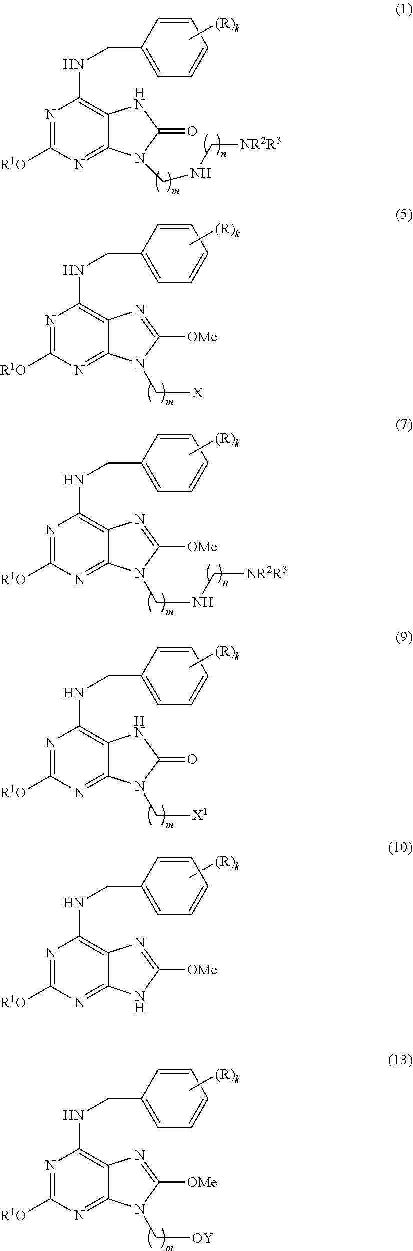

wherein k, m, n, R, R¹, R², R³, X, X¹ and Y are the same as defined above, or a salt thereof.
[12] A compound represented by the following formula (23) or (24):

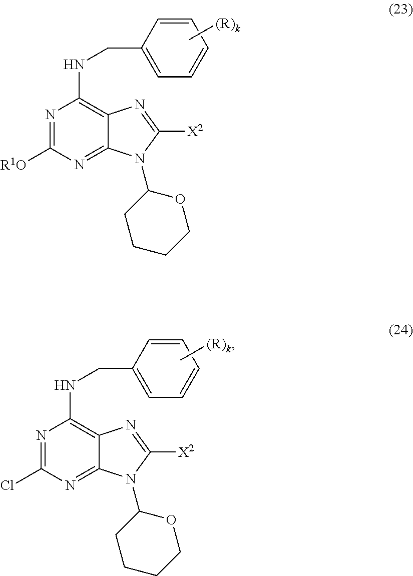

wherein X² is hydrogen atom, bromine atom or methoxy group, and k, R and R¹, are the same as defined above.

An intermediate of the present invention may fat hydrate and/or solvate and therefore, these hydrate and/or solvate are included in an intermediate of the present invention.

Furthermore, an intermediate represented by the formula (10) of the present invention may form a tautomer, if any and a tautomer thereof is included in an intermediate of the present invention.

### EFFECT OF INVENTION

According to the present invention, it has become possible to provide a novel method for preparing an adenine compound represented by the formula (4) or a pharmaceutically acceptable salt thereof which is useful as a medicament and an intermediate for preparing compound (4). According to the present invention, it is not required for a high pressure-reaction apparatus using ammonia gas required in the known methods. Furthermore, as an intermediate of the present invention has benzyl group on amino group at position 6, the intermediate does not show any pharmacological activity and therefore the object compound can be safely prepared in a more simple apparatus.

### THE BEST MODE FOR CARRYING OUT THE PRESENT INVENTION

The present invention is explained more in detail below.

In the present specification, “halogen atom” includes fluorine atom, chlorine atom, bromine atom and iodine atom, preferably fluorine atom and chlorine atom.

In the present specification, “C₁₋₆ alkyl group” includes C₁₋₆ streight or branched alkyl group, such as methyl group, ethyl group, propyl group, 1-methylethyl group, butyl group, pentyl group, hexyl group, etc.

In the present specification, “C₁₋₆ alkyloxy group” includes hydroxyl group substituted by C₁₋₆ streight or branched alkyl group such as methoxy group, ethoxy group, propoxy group, 1-methylethoxy group, butoxy group, pentyloxy group, hexyloxy group, etc.

In the present specification, “C₁₋₃ alkyl group” includes methyl group, ethyl group, propyl group, and 1-methylethyl group.

In the present specification, m and n are independently preferably an integer of 2 to 4, more preferably 3.

In the present specification, k is 1 or 2.

In the present specification, R² and R³ are preferably the same or different, and C₁₋₆ alkyl group, or R² and R³ are preferably combined with an adjacent nitrogen atom to form pyrrolidine, morpholine, thiomorpholine, piperidine, homopiperidine, piperazine or homopiperazine. Nitrogen atom of position 4 of said piperazine or homopiperazine may be substituted by C₁₋₄ alkyl group. More preferably R² and R³ are combined with an adjacent nitrogen atom to form a morpholine.

In the present specification, R⁴ is preferably methyl group.

Each step relating to the method of the present invention is explained

### Step (a)

In step (a) of above item [1], the salts of a compound represented by the formula (1) and a compound represented by the formula (2) include such as hydrochloride, hydrobromide, maleate, fumarate, oxalate, and so on.

The debenzylation reaction of a compound represented by the formula (1) to prepare a compound represented by the formula (2) is carried out by (a1) hydrogenation using hydrogen gas, formic acid or ammonium formate in the presence of a palladium-carbon catalyst or hydroxypalladium-carbon catalyst, by (a2) by debenzylation using alkyl chloroformate or substituted alkyl chloroformate, and so on. Alkylchloroformate or substituted alkylchloroformate includes for example 1-chloroethylchloroformate, 2,2,2-trichloroethyl chloroformate, 2-(trimethylsilyl)ethylchloroformate, vinylchloroformate, etc. Each reaction condition is known, and for example can be referred to J. Org. Chem., 52, 19 (1987), Tetrahedron Lett., 28, 2331 (1987), J. Org. Chem., 49, 2081 (1984), Tetrahedron Lett., 27, 3979 (1986), Tetrahedron Lett., 1567 (1977), etc.

### Step (b)

A boron-containing reducing agent used in step (b) of above item [1] includes for example, sodium triacetoxyborohydride, sodium cyanoborohydride, dimethylsulfideboron complex, 2-pycolin-boron complex, etc., preferably sodium triacetoxyborohydride, sodium cyanoborohydride, more preferably sodium triacetoxyborohydride.

In case of a salt of compound (2), this reaction can be conducted under neutral or acidic condition in the presence of a base equimolar or less to compound (2) or in the absence of the base. Furthermore, compound (2) and compound (3) are stirred in the presence a base more than equimolar to compound (2), and then the mixture is adjusted to acidic and may be reacted with a boron-containing reducing agent. The latter method is preferable. The base includes for example, an organic amine such as triethylamine, diisopropylethylamine, dimethyaminopyridine, preferably triethyamine. The reductive amination of compound (2) and compound (3) is usually under acidic condition such as in the presence of acetic acid, etc.

The reaction temperature is 15 to 40° C., preferably 20 to 30° C.

The reaction solvent is not specially limited, but is N-methylpyrrolidone (NMP), dimethyformamide (DMF), dimethy sulfoxide (DMSO), dichloromethane, tetrahydrofuran, etc., preferably N-methylpyrrolidone (NMP), dimethylformamide (DMF), more preferably N-methylpyrrolidone (NMP). In case of using triethylamine as a base, N-methylpyrrolidone (NMP) as a solvent is especially preferably used.

The reaction time is usually 3 to 24 hours.

Compound (4) can be isolated in a free form or a salt with an appropriate acid. The salt is not specially limited as long as it is a pharmaceutically acceptable non-toxic salt, but includes hydrochloride, sulfate, hydrobromide, maleate, fumarate, nitrate, orthophosphate, acetate, benzoate, methansulfonate, ethanesulfonate, L-lactate, aspartate, 2-naphthalenesulfonate, citrate, 1,5-naphthalenedisulfonate, succinate, oxalate, etc.

### Step (c)

In step (c) of above item [4], the salts of a compound represented by the formula (6) include preferably hydrochloride, hydrobromide, and the like.

Amount of compound (6) used in this reaction is 1 to 20 mole equivalents, preferably 5 to 10 mole equivalents to compound (5).

This reaction can be conducted in the presence or absence of a base. In case of a salt of compound (6), a base being at least equimolar amount to the salt of compound (6) must be added. The base includes for example, an organic amine such as triethylamine, diisopropylethylamine, 2,6-lutidine, dimethyaminopyridine, etc., preferably triethyamine.

The reaction temperature is not specially limited, but is usually 15 to 40° C., preferably 25 to 35° C.

The reaction solvent is not specially limited, but is N-methylpyrrolidone (NMP), N,N-dimethylformamide (DMF), dimethy sulfoxide (DMSO), etc., preferably N-methylpyrrolidone (NMP). The reaction may be preferably carried out without a solvent.

The reaction time is usually 3 to 24 hours.

The salt of compound (7) is not specially limited, but is hydrochloride, hydrobromide, maleate, fumarate, oxalate, etc.

### Step (d)

In step (d) of above item [4], as the acid used in a conversing process from compound (7) into compound (1) is illustrated a strong acid such as hydrochloric acid, hydrobromic acid, sulfuric acid, methanesulfonic acid, toluenesulfonic acid, etc, preferably hydrochloric acid, hydrombromic acid. The concentration of hydrochloric acid or hydrobromic acid is 0.1M to 12M, preferably 1M to 6 M.

The reaction in this step can be carried out without a solvent, but usually after dissolving compound (7) in a suitable organic solvent, to the solution is added the acid.

The organic solvent is not specially limited as long as compound (7) dissolves therein, but includes for example, methanol, toluene, THF, 1,4-dioxane, a mixture thereof, preferably a mixture of toluene and THF.

The reaction temperature is preferably 0 to 40° C., preferably 20 to 30° C.

The reaction time is usually 1 to 5 hours.

The acid is usually added to the reaction mixture in the above step for preparing compound (7) or to a solution containing compound (7) extracted from the reaction mixture is added the acid to prepare compound (1) from compound (7).

The acid can be used, if necessary after dissolving it in water or an organic solvent. For example, in case of hydrochloric acid or hydrobromic acid, the acid may be further diluted with water or may be dissolved in an organic solvent such as ethanol or dioxane.

### Step (e)

Step (e) of above item [5], namely the conversion-step of compound (8) into compound (9) is carried out by the same method as the above step (d).

### Step (f)

Step (f) of above item [5], namely the conversion-step of compound (9) into compound (1) is carried out by the same method as the above step (c).

### Step (g)

In step (g) of above item [7], condensation reaction of compound (10) and compound (11) is usually carried out in the presence of a base, such as an inorganic base like potassium carbonate, sodium carbonate, etc.

The reaction temperature is 20 to 60° C., preferably 20 to 30° C.

A reaction solvent is not specially limited, but is N-methylpyrrolodone (NMP), dimethyl sulfoxide (DMSO), dimethylformamide (DMF), chloroform, dichloromethane, etc.

The reaction time is usually 2 to 8 hours.

Compound (8) produced in this reaction can be isolated or can be subjected to next reaction step without isolation.

### Step (h)

Step (h) of above item [8], namely the conversion-step into compound (13) by condensation reaction of compound (10) and compound (12) is carried out by the same method as the above step (g).

In case that Y is a protective group of hydroxy group in compound (12) and compound (13), the protective group is not specially limited, but the protective group described in “Protective Groups in Organic Synthesis 3rd Edition (John Wiley & Sons, Inc.; 1999)” and the like can be used. For example, an ester-protective group such as acetyl group, formyl, etc., and a silyl ether protective group such as trimethylsilyl, triethylsilyl, t-butyldimethylsilyl, etc. are illustrated.

### Step (i)

Step (i) of above item [8] is carried out according to the well known deprotection reaction described in “Protective Groups in Organic Synthesis 3rd Edition (John Wiley 86 Sons, Inc.; 1999)”, and the like. For example, when Y is an ester-protective group, deprotection reaction can be carried out according to hydrolysis of ester known in the art. For example, alkali-hydrolysis is conducted using an alkali metal hydroxide such as sodium hydroxide, potassium hydroxide, etc. Furthermore, acid-hydrolysis is conducted using an acid such as hydrochloric acid, sulfuric acid, etc.

The reaction temperature is about 20 to 100° C.

The reaction solvent is not specially limited, but a conventional organic solvent used in hydrolysis of ester can be used. Hydrolysis can be carried in an aqueous solution of alkali metal hydroxide, an aqueous hydrochloric acid solution or an aqueous sulfulic acid solution without using an organic solvent. Example of the solvent is 1,4-dioxane-water, THF-water, or water-alcohol, preferably methanol-water.

The reaction time is usually 1 to 24 hours.

When Y is a silyl-ether protective group, the deprotection is conducted according to a desilylation known in the art. For example, the desilylation can be easily and in good yield conducted by using trifruoroacetic acid, hydrochloric acid, tertabutylammonium fluoride (TBFA), hydrofluoric acid (HF), cesium fluoride (CsF).

Compound (14) produced in this reaction can be isolated can be subjected to next reaction step without isolation.

### Step (j)

In step (j) of above item [8], compound (14) is usually methanesulfonylated with methanesulfonyl chloride to prepare compound (15).

Methanesulfonylation is usually in the presence of a base, such as an organic base like triethylamine, diisopropylethylamine, pyridine, etc., preferably triethylamine or diisopropylethylamine.

Further more, trimethylamine hydrochloride can be used as an agent for protecting a by-product, and dimethyaminopyridine can be used as a reaction promoting agent.

The reaction temperature is around 0° C. to 20° C.

The reaction solvent is not specially limited, but includes N-methylpyrrolidone (NMP), dimethyl sulfoxide (DMSO), dimethylformamide (DMF), dichloromethane, chloroform, tetrahydrofuran, etc.

The reaction time is usually 10 to 60 minutes.

### Step (k)

Compound (16) used as a starting material is known and can be commercially available or prepared accordance with a known method in the art.

In step (k) of above item [10], benzylamine derivative (17) includes benzylamine, 4-methoxybenzylamine, 2,4-dimethoxybenzylamine, 4-nitrobenzylamine and the like, preferably benzylamine.

The above reaction can be conducted in the presence or absence of a base. The base includes an organic base such as triethylamine, diisopropylethylamine, dimethyaminopyridine, and the like.

The reaction solvent includes an alcohol solvent such as methanol etc., an ether solvent such as tetrahydrofuran, etc., dimethylformamide (DMF), and the like.

The reaction temperature is selected from the range of about 20° C. to boiling point of the solvent.

The reaction time is usually 0.5 to 12 hours.

The known method for preparing compound (4) relates to prepare it without protecting amino group at position 6 of purine nucleus, and compound (4) was prepared starting from 2-chloro-9-(tetrahydro-2H-pyran-2-yl)-9H-purine-6-amine obtained by reacting compound (16) with ammonia under high pressure. However, according to the method of the present invention which comprises protecting amino group at position 6 with a protective group such as benzyl group, etc., an intermediate, compound (18) can be prepared under atmospheric pressure and therefore, the method has a great advantage from the viewpoint of being unnecessary for the reaction under high pressure.

### Step (1)

In step (1) of above item [10], the base includes an alkali metal such as sodium, potassium, etc., an alkali hydride such as sodium hydride, potassium hydride, etc., and the like.

The reaction solvent includes an ether solvent such as tetrahydrofuran etc., dimethylformamide (DMF) and the like. Compound (19) may be served as a solvent.

The reaction temperature is selected from the range of about 20° C. to boiling point of the solvent.

The reaction time is usually 0.5 to 12 hours.

### Step (m)

The solvent used in step (m) includes dichloromethane, chloroform, tetrahydrofuran, etc.

This reaction usually proceeds by dissolving compound (20) in the solvent, and then adding bromine thereto. In order to prevent a side reaction such as elimination of tetrahydropyrane ring, it is preferable to add an additive such as sodium acetate, sodium phosphate, etc., more preferably an aqueous sodium acetate solution.

The reaction temperature is selected from the range of about 10° C. to about 30° C.

The reaction time is usually 1 to 6 hours.

### Step (n)

In step (n) of above item [10], the base includes an alkali metal such as sodium, potassium, etc., an alkali hydride such as sodium hydride, potassium hydride, etc., an inorganic base such as sodium hydroxide, potassium hydroxide, etc., and the like.

The reaction solvent includes methanol, an ether solvent such as tetrahydrofuran, etc., dimethylformamide (DMF), water, a mixture thereof, and the like.

The reaction temperature is selected from the range of about 20° C. to point of the solvent.

### Step (o)

In step (o) of above item [10], the acid used in this step includes hydrochloric acid, hydrobromic acid, sulfuric acid, methanesulfonic acid, trifluoroacetic acid, etc, preferably trifluoroacetic acid.

The reaction solvent includes an alcohol such as methanol, an ether solvent such as tetrahydrofuran, and the like.

The reaction time is selected from the range of about 10° C. to about 30° C.

The reaction time is usually 1 to 24 hours.

The present invention is explained by following examples, but the invention is not limited by them.

In the following examples, reaction agents and solvents commercially available were used. Organic solutions were dried over anhydrous sodium sulfate unless there is a specific definition.

Chemical shift of ¹H NMR was reported on the basis of inner standard tetramethysilan.

Me means methyl group, and TFA means trifluoroacetic acid in following formulas.

### Example 1

### N-Benzyl-2-chloro-9-(tetrahydro-2H-pyran-2-yl)-9H-purine-6-amine

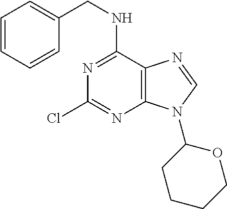

To a suspension of 2,6-dichloro-9-(tetrahydro-2H-pyran-2-yl)-9H-purine (10.00 g, 36.61 mmol) in methanol (100 ml) was added benzylamine (8.24 g, 76.89 mmol) and the mixture was refluxed under heating for 0.5 hour. The reaction mixture was cooled to room temperature and thereto was added water (200 ml). The resulting crystal was filtered to give the subject compound (12.38 g, 98%).

¹H NMR δ (CDCl₃) 8.10 (1H, s), 7.42-7.26 (6H, m), 5.72 (1H, dd, J=11.3, 2.4 Hz), 4.8 (2H, s), 4.20-4.16 (1H, m), 3.81-3.73 (1H, m), 2.21-1.66 (6H, m).

### Example 2

### N-Benzyl-2-butoxy-9-(tetrahydro-2H-pyran-2-yl)-9H-purine-6-amine

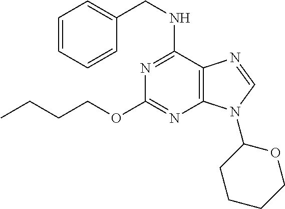

The compound (8.00 g, 23.27 mmol) prepared by example 1 was dissolved in a sodium butoxide-butanol solution prepared with butanol (80 ml) and metal sodium (1.60 g, 69.81 mmol), and the solution was stirred for 2 hours at 120° C. After being cooled to room temperature, the solvent was removed and to the residue was added water, followed by extraction with toluene. The organic layer was washed with water, dried and concentrated in vacuo. To the residue was added toluene-hexane to crystallize. The resulting crystal was filtered to give the subject compound (8.03 g, 94%).

¹H NMR δ (CDCl₃) 7.61 (1H, s), 7.36-7.22 (5H, s), 6.86 (1H, bs), 5.62 (1H, dd, J=11.3, 2.4 Hz), 4.81 (2H, s), 4.33 (2H, t, J=6.8 Hz), 4.13-4.09 (1H, m), 3.76-3.69 (1H, m), 2.04-1.44 (10H, m), 0.95 (3H, t, J=7.4 Hz).

### Example 3

### N-Benzyl-8-bromo-2-butoxy-9-(tetrahydro-2H-pyran-2-yl)-9H-purine-6-amine

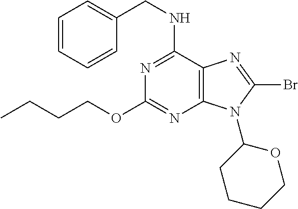

To a solution of the compound (18.70 g, 49.02 mmol) prepared by example 2 in ethyl acetate (935 ml) were added sodium acetate (9.70 g, 118.27 mmol) and water (1.87 ml), and to the solution was dropped bromine (15.67 g, 98.04 mmol), followed by stirring for 1 hour at room temperature. After adding a 10% aqueous sodium thiosulfate solution, the solution was separated with a separating funnel. The organic layer was washed with an aqueous sodium hydrogencarbonate solution, dried and concentrated in vacuo. The residue was purified with silica gel chromatography (ethyl acetate:hexane=3:1) to give the subject compound (22.35 g, 99%).

¹H NMR δ (CDCl₃) 7.34-7.21 (5H, m), 5.94 (1H, bs), 5.62 (2H, dd, J=11.3, 2.4 Hz), 4.77 (2H, s), 4.33 (2H, t, J=6.8 HZ), 4.18-4.14 (1H, m), 3.73-3.66 (1H, m), 3.03-2.96 (1H, m), 2.10-1.44 (9H, m), 0.96 (3H, t, J=7.4 Hz).

### Example 4

### N-Benzyl-2-butoxy-8-methoxy-9-(tetrahydro-2H-pyran-2-yl)-9H-purine-6-amine

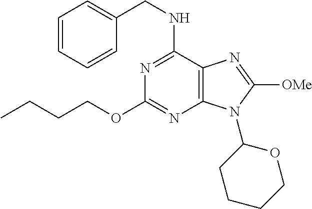

To a solution of the compound prepared by example 3 (22.30 g, 48.44 mmol) in methanol (558 ml) was 5N aqueous sodium hydroxide (112 ml) solution, and the solution was refluxed under heating for 3 hours. After adding water, methanol was removed therefrom in vacuo, followed by extracting with toluene. The organic layer was washed with water, dried and concentrated in vacuo to give the subject compound (20.04 g, quant.).

¹H NMR δ (CDCl₃) 7.37-7.26 (5H, m), 5.63 (1H, bs), 5.52 (1H, dd, J=11.3, 2.4 Hz), 4.80 (2H, d, J=4.8 Hz), 4.30 (2H, t, J=6.8 Hz), 4.12-4.09 (1H, m), 4.07 (3H, s), 3.72-3.65 (1H, m), 2.76-2.71 (1H, m), 2.04-1.43 (9H, m), 0.95 (3H, t, J=7.4 Hz).

### Example 5

### N-Benzyl-2-butoxy-8-methoxy-9H-purine-6-amine.trifluoroacetate

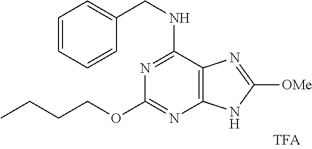

To a solution of the compound prepared by example 4 (20.00 g, 48.60 mmol) in methanol (500 ml) was dropped trifluoroacetic acid (100 ml), and the mixture was stirred for 24 at room temperature. After concentrated in vacuo, to the residue was added ethyl acetate-hexane to crystallize. The crystal was filtered to give the subject compound (16.13 g, 75%).

¹H NMR δ (DMSO-d₆) 7.93 (1H, bs) 7.37-7.22 (5H, m), 4.68 (2H, s), 4.24 (2H, t, J=6.8 Hz), 4.03 (3H, s), 1.67-1.60 (2H, m), 1.39-1.33 (2H, m) 0.89 (3H, t, J=7.3 Hz).

### Example 6

### N-Benzyl-2-butoxy-9-(3-chloropropyl)-8-methoxy-9H-purine-6-amine

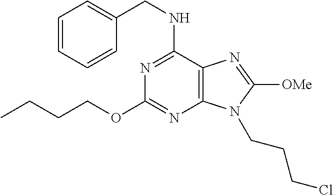

To a solution of the compound prepared by example 5 (15.00 g, 33.98 mmol) in dimethyl formamide (150 ml) was added sodium carbonate (14.09 g, 101.95 mmol), and then thereto was added 1-bromo-3-chloropropane (10.70 g, 67.97 mmol). The mixture was stirred at room temperature for 4 hours. The reaction mixture was concentrated in vacuo and after adding water to the residue, the mixture was extracted with toluene. The organic layer was washed with water, dried and concentrated in vacuo. The residue was purified with silica gel chromatography (ethyl acetate:hexane=3:1) to give the subject compound (12.05 g, 88%).

¹H NMR δ (CDCl₃) 7.37-7.24 (5H, m), 5.76 (1H, t, J=5.6 Hz), 4.80 (2H, d, J=5.6 Hz), 4.30 (2H, t, J=6.8 Hz), 4.07 (2H, t, J=6.8 Hz), 4.06 (3H, s), 3.52 (2H, t, J=6.5 Hz), 2.27-2.20 (2H, m), 1.80-1.73 (2H, m), 1.50-1.42 (2H, m), 0.95 (3H, t, J=7.4 Hz).

### Example 7

### (6-Benzylamino)-2-butoxy-9-(3-chloropropyl)-7,8-dihydro-8H-purine-8-one

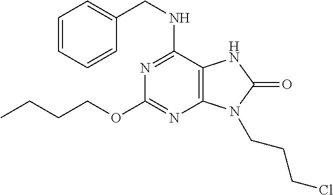

To a solution of the compound prepared by example 6 (12.00 g, 29.71 mmol) in methanol (240 ml) was added 4N HCl-dioxane (60 ml) and the mixture was stirred at room temperature for 12 hours. After removal of the solvent in vacuo, to the residue was added water. The solution was neutralized with an aqueous ammonium solution and the resulting crystal was filtered to give the subject compound (10.08 g, 87%).

¹H NMR δ (DMSO-d₆) 9.81 (1H, bs), 7.36-7.26 (5H, m), 7.01 (1H, t, J=5.7 Hz), 4.61 (2H, d, J=5.7 Hz), 4.16 (2H, t, J=6.7 Hz), 3.81 (2H, t, J=6.8 Hz), 3.66 (2H, t, J=6.4 Hz), 2.14-2.07 (2H, m), 1.66-1.58 (2H, m), 1.39-1.33 (2H, m), 0.90 (3H, t, J=7.4 Hz).

### Example 8

### (6-Benzylamino)-2-butoxy-9-{3-[(3-morpholin-4-ylpropyl)amino]propyl}-7,9-dihydro-8H-purine-8-one

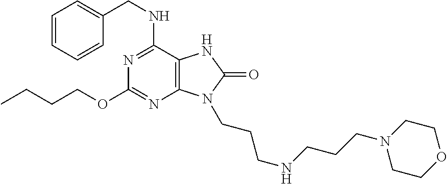

To a solution of the compound prepared by example 7 (5.00 g, 12.82 mmol) in dimethylformamide (5 ml) was added 3-morphlinopropylamine (18.50 g, 128.24 mmol), and the mixture was stirred at 80° C. for 3 hours. Thereto was added ethyl acetate and the mixture was washed three times with saturated brine. The organic layer was dried and concentrated in vacuo to give the subject compound (5.92 g, 93%).

¹H NMR δ (CDCl₃) 7.33-7.21 (5H, m), 6.92 (1H, t, J=5.6 Hz), 4.77 (2H, d, J=5.6 Hz), 4.25 (2H, t, J=6.8 Hz), 3.72-3.65 (2H, m), 3.68 (2H, t, J=6.8 Hz), 3.58 (2H, t, J=6.4 Hz), 2.57-2.31 (10, m), 1.78-1.62 (6H, m), 1.42-1.38 (2H, m), 0.93 (3H, t, J=7.4 Hz).

### Example 9

### 6-Amino-2-butoxy-9-{3-[(3-morpholin-4-ylpropyl)amino]propyl}-7,9-dihydro-8H-purine-8-one-trihydrochloride

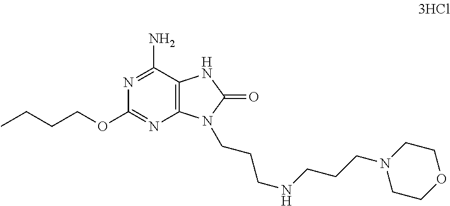

To a solution of the compound prepared by example 8 (5.92 g, 11.90 mmol) in methanol (120 ml) were added hydrochloric acid (12 ml) and Pd—C (6.00 g, 50% wet), and the mixture was stirred under hydrogen atomosphea for 2.5 hours at room temperature. The reaction mixture was filtered with Celite, and the filtrate was concentrated in vacuo. To the residue were added methanol (60 ml) and 2-propanol (120 ml) and the mixture was stirred. The resulting crystal was filtered to give the subject compound (4.72 g, 77%).

¹H NMR δ (DMSO-d₆) 11.12 (1H, bs), 10.58 (1H, bs), 8.93 (2H, bs), 4.20 (2H, t, J=6.6 Hz), 3.97-3.94 (2H, m), 3.84-3.74 (4H, m), 3.40 (1H, bs), 3.38 (1H, bs), 3.19-3.14 (2H, m), 3.07-2.92 (6H, m), 2.10-1.91 (4H, m), 1.69-1.62 (2H, m), 1.45-1.35 (2H, m), 0.92 (3H, t, J=7.4 Hz).

### Example 10

### Methyl (3-{[[3-(6-amino-2-butoxy-8-oxo-7,8-dihydro-9H-purine-9-yl)propyl](3-morpholin-4-ylpropyl)amino]methyl}phenyl acetate

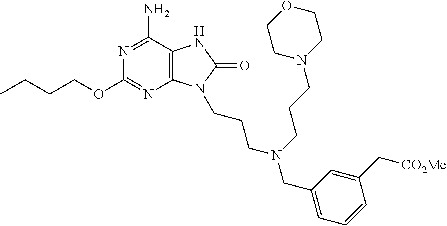

To a solution of the compound prepared by example 9 (1.00 g, 1.93 mmol) in N-methyl-2-pyrrolidine (10 ml) were added triethylamine (1.14 ml, 7.73 mmol) and methyl (3-formylphenyl)acetate (0.52 g, 2.90 mmol), and the mixture was stirred at room temperature for 0.5 hour. Thereto was added sodium triacetoxyborohydride (0.82 g, 3.87 mmol), and the mixture was further stirred for 24 hours. Thereto was added water and the solution was adjusted to pH8 with a diluted aqueous ammonium solution. The resulting crystal was filtered to give the subject compound (0.98 g, 89%).

¹H NMR δ (DMSO-d₆) 9.82 (1H, bs), 7.24-7.15 (3H, m), 7.10 (1H, d, J=7.3 Hz), 6.39 (2H, bs), 4.11 (2H, t, J=6.6 Hz), 3.67 (2H, t, J=7.2 Hz), 3.64 (2H, s), 3.59 (3H, s), 3.46-3.49 (6H, m), 2.41-2.34 (4H, m), 2.22-2.16 (6H, m), 1.85-1.80 (2H, m), 1.65-1.57 (2H, m), 1.52-1.45 (2H, m), 1.40-1.31 (2H, m), 0.89 (3H, t, J=7.4 Hz).

### INDUSTRIAL APPLICABILITY

The present invention relates to a method for preparing an adenine compound useful as a medicament, and to an intermediate for preparing it.

1. A method for preparing a compound represented by the following formula (4): 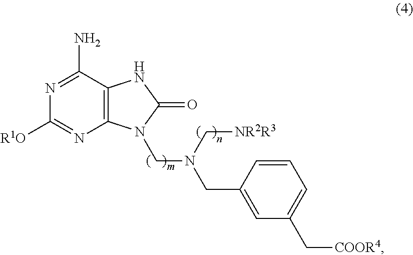
wherein m and n are independently an integer of 2 to 5, R¹ is C₁₋₆ alkyl group, R² and R³ are the same or different, and hydrogen atom, or C₁₋₆ alkyl group, or R² and R³ are combined with an adjacent nitrogen atom to form pyrrolidine, morpholine, thiomorpholine, piperidine, homopiperidine, piperazine or homopiperazine, and nitrogen atom of position 4 of said piperazine or homopiperazine may be substituted by C₁₋₄ alkyl group, and R⁴ is C₁₋₃ alkyl group, or a pharmaceutically acceptable salt,
which comprises
step (a) for preparing a compound represented by the following compound (2): 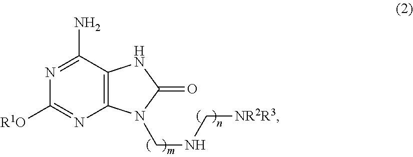
wherein m, n, R¹, R² and R³ are the same as defined above, or its salt
which comprises subjecting a compound represented by the following formula (1): 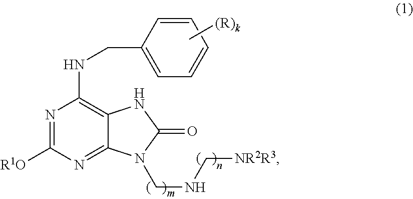
wherein k is an integer of 1 or 2, R is hydrogen atom, halogen atom, C₁₋₆ alkyl group, C₁₋₆ alkyloxy group or nitro group, and when k is 2, R is the same or different, and R¹, R², R³, m and n are the same as defined above, or a salt thereof to debenzylation reaction, and then
step (b) for preparing a compound (4) or its pharmaceutically acceptable salt which comprises reacting compound (2) or its salt prepared in the above step (a) or a salt thereof and a compound represented by the formula (3): 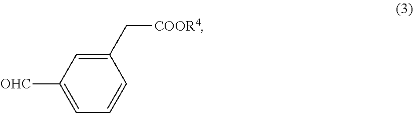
wherein R⁴ is C₁₋₃ alkyl group,
in the presence of boron-containing reducing agent.

2. The method according to claim 1 wherein the debenzylation reaction is carried out (a1) by hydrogenation using hydrogen gas, formic acid or ammonium formate in the presence of a palladium-carbon catalyst or hydroxypalladium-carbon catalyst, or (a2) by debenzylation using alkyl chloroformate or substituted alkyl chloroformate.

3. The method according to claim 1 or 2 wherein boron-containing reducing agent in step (b) is sodium triacetoxybolohydride.

4. The method according to claim 1 wherein the method contains further process for preparing compound (1) or its salt, which comprises
steps (c) for preparing a compound represented by the following formula (7): 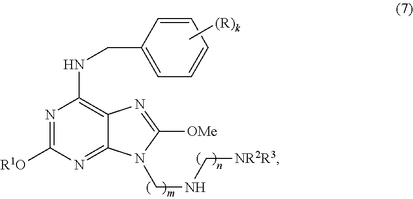
wherein k, m, n, R, R¹, R² and R³ are the same as defined above, or a salt thereof, which comprises
reacting a compound represented by the formula (5): 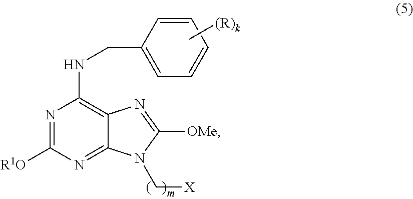
wherein X is chlorine atom, bromine atom or methanesulfonyloxy group, k, m, R and R¹ are the same as defined above,
and a compound represented by the formula (6): 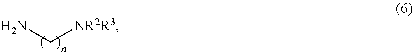
wherein n, R² and R³ are the same as defined above,
or a salt thereof, and then
step (d) for preparing compound (1) which comprises treating compound (7) or its salt with an acid.

5. The method according to claim 1 wherein the method contains further process for preparing compound (1) or its salt, which comprises
step (e) for preparing a compound represented by the following formula: 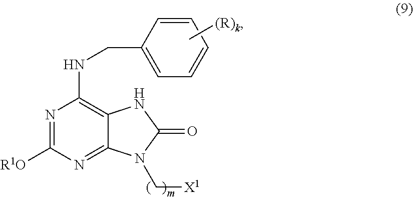
wherein k, m, R, R¹ and X¹ are the same as defined above,
which comprises treating a compound represented by the following formula (8): 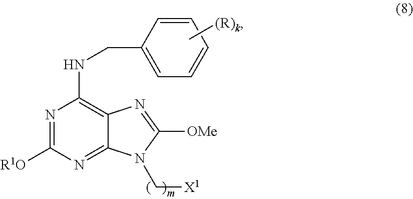
wherein X¹ is chlorine atom or bromine atom and k, m, R and R¹ are the same as defined above,
with an acid, and then
step (f) for preparing compound (1) or its salt which comprises reacting a compound (9) obtained above with a compound represented by the formula (6): 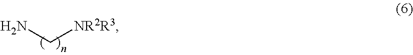
wherein n, R² and R³ are the same as define above,
or its salt.

6. The method according to claim 4 or 5 wherein the acid used in step (d) or (e) is an acid or acids selected from hydrochloric acid, hydrobromic acid, sulfuric acid, methanesulfonic acid and p-toluenesulfonic acid.

7. The method according to claim 4 wherein the method contains further process for preparing (8) or its salt, which comprises step (g) for preparing compound (8) which compresses reacting a compound represented by the formula (10): 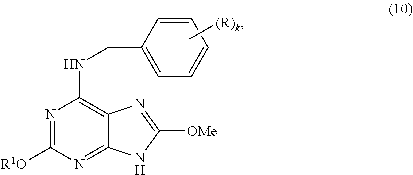
wherein k, R and R¹ are the same as defined above,
or its salt with a compound represented by the formula (11): 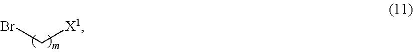
wherein m and X¹ are the same as defined above,
in the presence of a base.

8. The method according to claim 4 wherein the method contains further process for preparing a compound represented by the following formula (15): 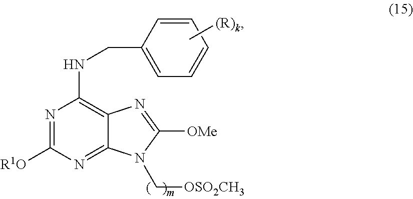
wherein k, m, R and
R¹ are the same as defined above,
or its salt, which comprises
step (h) for preparing a compound represented by the following formula (13): 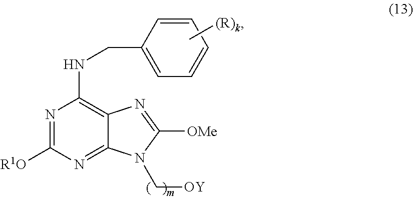
wherein Y is hydrogen atom or a protective group of hydroxy group and k, m, R and R¹ are the same as defined above,
which comprises reacting a compound represented by the following formula (10): 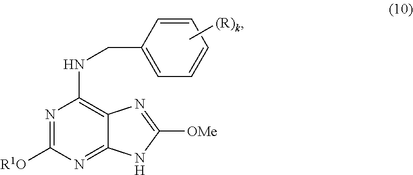
wherein k, R and R¹ are the same as defined above,
or its salt with a compound represented by the following formula (12): 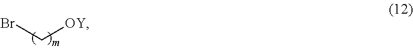
wherein Y and m is the same as defined above,
in the presence of a base, and then
step (i) for preparing a compound represented by the following formula (14): 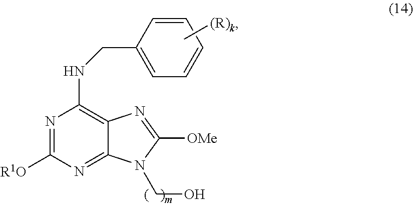
wherein k, m, R and R¹ are the same as defined above,
which comprises subjecting compound (13) wherein Y is a protecting group of hydroxy group to deprotection reaction, and then
step (j) for preparing compound (15) which comprises reacting compound (14) prepared by step (h) or (i) with methanesulfonyl chloride in the presence of a base.

9. The method according to claim 7, 8 or 13 wherein the method contains further process for preparing compound (10) or its salt, which comprises step (k) for preparing a compound represented by the following formula (18): 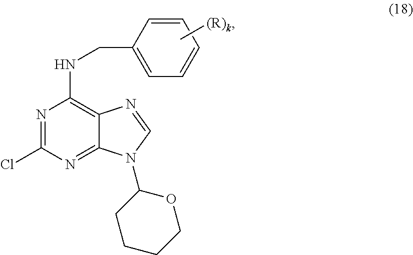
wherein k and R are the same as defined above,
which comprise reacting a compound represented by the following formula (16): 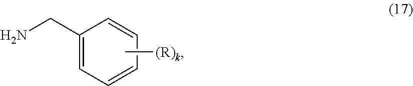
with a compound represented by the formula (17): 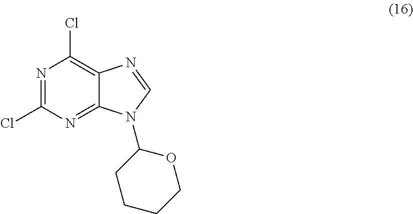
wherein k and R are the same as defined above,
or its salt, and then
step (1) for preparing a compound represented by the following formula (20): 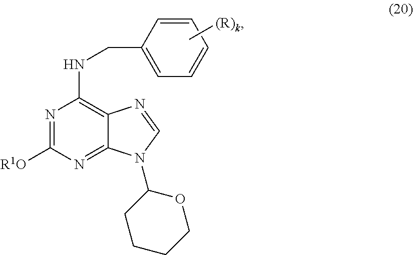
wherein k, R and R¹ are the same as define above,
which comprises reacting compound (18) prepared in step (k) with a compound represented by the following formula (19):
R¹—OH (19),
wherein R¹ is the same as defined above,
in the presence of a base, and then
step (m) for preparing a compound represented by the formula (21): 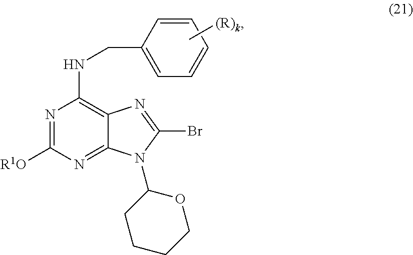
wherein k, R and R¹ are the same as defined above,
which comprises reacting compound (20) prepared in step (1) with bromine in the presence of sodium acetate or sodium phsphate, or in the absence of said salt and then step (n) for preparing a compound represented by the following formula (22): 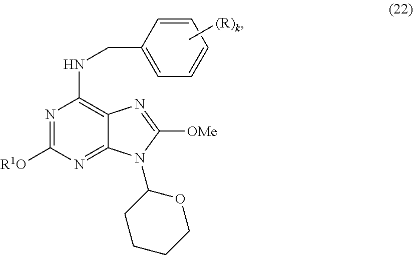
wherein k, R and R¹ are the same as defined above,
which comprises reacting compound (21) prepared in step (m) with methanol in the presence of a base, and then
step (o) for preparing compound (10) which comprises treating compound (22) prepared in step (n) with an acid.

10. The method according to claim 9 wherein the acid used in step (o) is trifluoroacetic acid.

11. A compound selected from compounds represented by following formulas (1). (5), (7), (9), (10) and (13): 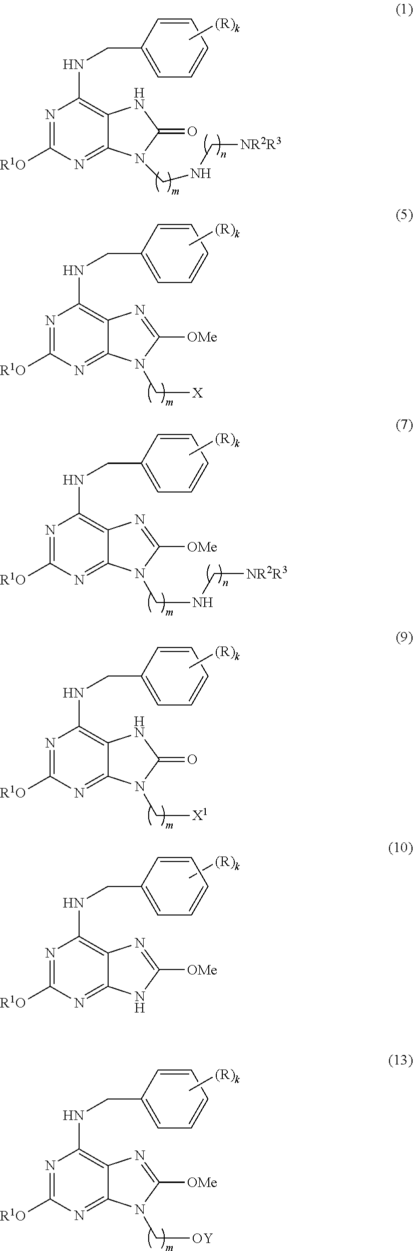
wherein k, m, n, R, R¹, R², R³, X, X¹ and Y are the same as defined above, or a salt thereof.

12. A compound represented by the following formula (23) or (24): 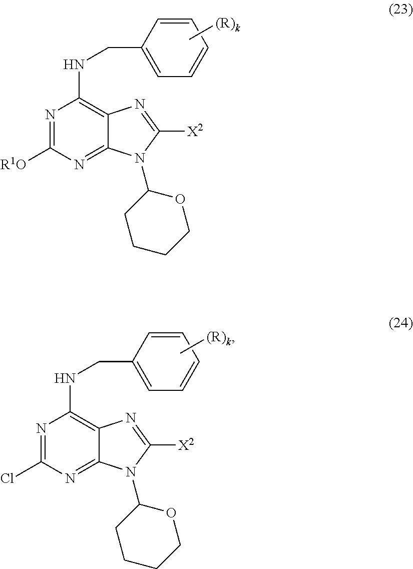
wherein X² is hydrogen atom, bromine atom or methoxy group, and k, R and R¹ are the same as defined above.

13. The method according to claim 5 wherein the method contains further process for preparing (8) or its salt, which comprises
step (g) for preparing compound (8) which compresses reacting a compound represented by the formula (10): 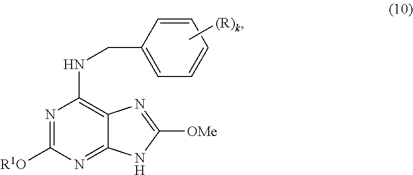
wherein k, R and R¹ are the same as defined above,
or its salt with a compound represented by the formula (11): 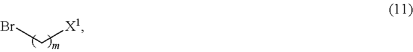
wherein m and X¹ are the same as defined above,
in the presence of a base.
